(19) United States
(12) Patent Application Publication
(10) Pub. No.: US 2023/0043660 A1
(43) Pub. Date: Feb. 9, 2023

(54) OSTIAL STENT DELIVERY DEVICE, SYSTEM, AND METHOD

(71) Applicant: BOSTON SCIENTIFIC SCIMED, INC., MAPLE GROVE, MN (US)

(72) Inventors: Javier Palomar-Moreno, Galway (IE); John Kilcooley, Galway (IE); Michelle Hannon, Galway (IE)

(73) Assignee: BOSTON SCIENTIFIC SCIMED, INC., MAPLE GROVE, MN (US)

(21) Appl. No.: 17/880,126

(22) Filed: Aug. 3, 2022

Related U.S. Application Data
(60) Provisional application No. 63/229,424, filed on Aug. 4, 2021.

(51) Int. Cl. A61F 2/958; A61M 25/10

(52) U.S. Cl. CPC A61F 2/958; A61M 25/1011; A61M 25/1002; A61F 2002/821

(57) ABSTRACT

An ostial stent delivery device may include a first elongate shaft including a first lumen and extending along a central longitudinal axis, wherein the first elongate shaft includes a first inflatable balloon fixedly attached proximate a distal end of the first elongate shaft; and a second elongate shaft including a second lumen, the first elongate shaft being at least partially disposed within the second lumen. The second elongate shaft includes a second inflatable balloon fixedly attached proximate a distal end of the second elongate shaft. The second inflatable balloon is disposed at least partially proximal of the first inflatable balloon. The first inflatable balloon has a substantially cylindrical shape along a majority of its length in the deployed configuration. The second inflatable balloon has a substantially biconical shape having a central axis oriented parallel to the central longitudinal axis in the deployed configuration.

### CROSS-REFERENCE TO RELATED APPLICATIONS

This application claims the benefit of priority of U.S. Provisional Application No. 63/229,424 filed Aug. 4, 2021, the entire disclosure of which is hereby incorporated by reference.

### TECHNICAL FIELD

The present disclosure pertains to medical devices, and methods for manufacturing and/or using medical devices. More particularly, the present disclosure pertains to an ostial stent delivery device, system, and/or method.

### BACKGROUND

A wide variety of intracorporeal medical devices have been developed for medical use, for example, intravascular use. Some of these devices include stent, catheters, medical device delivery systems (e.g., for stents, grafts, replacement valves, etc.), and the like.

Ostial stents may be used to improve and/or maintain patency within an ostium of a vessel, such as a coronary artery. In some treatments, the ostial stent may be used in conjunction with another procedure and/or medical device. For example, an ostial stent may be used along with a replacement heart valve implant in different techniques including, but not limited to, a protected transcatheter aortic valve replacement (TAVR) procedure and/or a chimney technique. Deployment of an ostial stent may involve multiple balloon catheters being exchanged to facilitate expansion of different portions of the ostial stent, thereby prolonging the procedure and/or introducing opportunities for error and/or injury to the patient. Of the known medical devices and methods, each has certain advantages and disadvantages. There is an ongoing need to provide alternative medical devices as well as alternative methods for manufacturing and/or using medical devices.

### SUMMARY

In one example, an ostial stent delivery device may comprise a first elongate shaft including a first lumen extending therein, the first elongate shaft extending along a central longitudinal axis, wherein the first elongate shaft includes a first inflatable balloon fixedly attached thereto proximate a distal end of the first elongate shaft, the first inflatable balloon being in fluid communication with the first lumen; and a second elongate shaft including a second lumen extending therein, the first elongate shaft being at least partially disposed within the second lumen. The second elongate shaft includes a second inflatable balloon fixedly attached thereto proximate a distal end of the second elongate shaft, the second inflatable balloon being in fluid communication with the second lumen. The second inflatable balloon is disposed at least partially proximal of the first inflatable balloon. The first inflatable balloon is configured to expand a distal portion of an ostial stent when the first inflatable balloon is shifted from a delivery configuration to a deployed configuration. The second inflatable balloon is configured to expand a proximal portion of the ostial stent when the second inflatable balloon is shifted from a delivery configuration to a deployed configuration. The first inflatable balloon has a substantially cylindrical shape along a majority of its length in the deployed configuration of the first inflatable balloon. The second inflatable balloon has a substantially biconical shape having a central axis oriented parallel to the central longitudinal axis in the deployed configuration of the second inflatable balloon.

In addition or alternatively to any example disclosed herein, when disposing the second inflatable balloon in the delivery configuration, a radially outermost extent of the second inflatable balloon is urged proximally and a proximal portion of the second inflatable balloon disposed proximal of the radially outermost extent of the second inflatable balloon is urged distally.

In addition or alternatively to any example disclosed herein, the second inflatable balloon is fixedly attached to the first inflatable balloon.

In addition or alternatively to any example disclosed herein, the second inflatable balloon is adhesively bonded to the first inflatable balloon.

In addition or alternatively to any example disclosed herein, the second inflatable balloon is welded to the first inflatable balloon.

In addition or alternatively to any example disclosed herein, the first elongate shaft includes a first proximal port in fluid communication with the first lumen and configured to receive a first inflation media for inflating the first inflatable balloon to the deployed configuration.

In addition or alternatively to any example disclosed herein, the second elongate shaft includes a second proximal port in fluid communication with the second lumen and configured to receive a second inflation media for inflating the second balloon to the deployed configuration.

In addition or alternatively to any example disclosed herein, the second inflatable balloon is oriented coaxially with the first inflatable balloon.

In addition or alternatively to any example disclosed herein, the first elongate shaft is disposed coaxially within the second lumen of the second elongate shaft.

In addition or alternatively to any example disclosed herein, an ostial stent system may comprise a guide catheter having a lumen extending therethrough, an ostial stent having a proximal portion and a distal portion, and an ostial stent delivery device slidably disposed within the lumen of the guide catheter. The ostial stent delivery device may comprise a first elongate shaft extending along a central longitudinal axis, the first elongate shaft having a first inflatable balloon fixedly attached thereto proximate a distal end of the first elongate shaft, and a second elongate shaft extending along the central longitudinal axis, the second elongate shaft having a second inflatable balloon fixedly attached thereto proximate a distal end of the second elongate shaft. The second inflatable balloon is disposed at least partially proximal of the first inflatable balloon. The distal portion of the ostial stent is secured to the first inflatable balloon in a collapsed configuration when the first inflatable balloon is disposed within the lumen of the guide catheter. The proximal portion of the ostial stent is secured to the second inflatable balloon in a collapsed configuration with the second inflatable balloon is disposed within the lumen of the guide catheter. When the first inflatable balloon and the second inflatable balloon are disposed outside of the lumen of the guide catheter in a first position, the distal portion of the ostial stent is in the collapsed configuration and the proximal portion of the ostial stent is in an expanded configuration.

In addition or alternatively to any example disclosed herein, when the first inflatable balloon and the second inflatable balloon are disposed outside of the lumen of the guide catheter in a second position, the distal portion of the ostial stent is shiftable to an expanded configuration upon inflation of the first inflatable balloon.

In addition or alternatively to any example disclosed herein, the first inflatable balloon is inflatable independently of the second inflatable balloon.

In addition or alternatively to any example disclosed herein, the first inflatable balloon is formed from a first material and the second inflatable balloon is formed from a second material.

In addition or alternatively to any example disclosed herein, in the expanded configuration, the proximal portion of the ostial stent has a substantially conical shape expanding radially outward in a proximal direction.

In addition or alternatively to any example disclosed herein, a method of delivering an ostial stent may comprise positioning a proximal portion of the ostial stent such that the proximal portion is the ostial stent is spaced apart proximal of an ostium of a vessel, expanding the proximal portion of the ostial stent to an expanded configuration while maintaining a distal portion of the ostial stent in a collapsed configuration, wherein the proximal portion of the ostial stent remains spaced apart from the ostium of the vessel, advancing the distal portion of the ostial stent into the vessel until the proximal portion of the ostial stent engages the ostium of the vessel, and expanding the distal portion of the ostial stent to an expanded configuration within the vessel to engage the distal portion of the ostial stent against a wall of the vessel.

In addition or alternatively to any example disclosed herein, prior to expanding the proximal portion of the ostial stent, the distal portion of the ostial stent is secured to a first inflatable balloon in the collapsed configuration and the proximal portion of the ostial stent is secured to a second inflatable balloon in a collapsed configuration.

In addition or alternatively to any example disclosed herein, expanding the proximal portion of the ostial stent includes inflating the second inflatable balloon without inflating the first inflatable balloon.

In addition or alternatively to any example disclosed herein, when the second inflatable balloon is inflated, at least a portion of the first inflatable balloon extends proximal of the ostium of the vessel.

In addition or alternatively to any example disclosed herein, expanding the distal portion of the ostial stent includes inflating the first inflatable balloon.

In addition or alternatively to any example disclosed herein, the second inflatable balloon includes a substantially biconical body portion having a radially outermost extent in a deployed configuration of the second inflatable balloon that is greater than a radially outermost extent of a substantially cylindrical body portion of the first inflatable balloon in a deployed configuration of the first inflatable balloon.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view illustrating aspects of an ostial stent delivery device;

FIGS. 2-5 illustrate an example technique for folding the second inflatable balloon of the ostial stent delivery device of FIG. 1;

FIG. 6 schematically illustrates aspects of an ostial stent system;

FIGS. 7-10 illustrate aspects of a method of delivering an ostial stent; and

FIGS. 11-14 illustrate aspects of a method of manufacturing an ostial stent delivery device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “transaortic valve implantation” and “transcatheter aortic valve implantation” may be used interchangeably and may each be referred to using the acronym “TAVI”. The terms “transaortic valve replacement” and “transcatheter aortic valve replacement” may be used interchangeably and may each be referred to using the acronym “TAVR”.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are medical devices that may be used within a portion of the cardiovascular system in order to diagnose, treat, and/or repair the system, for example during and/or in conjunction with a TAVI or TAVR procedure, or in place of a TAVI or TAVR procedure in patients not suitable for such. At least some of the medical devices disclosed herein may be delivered percutaneously and, thus, may be much less invasive to the patient, although other surgical methods and approaches may also be used. The devices disclosed herein may also provide a number of additional desirable features and benefits as described in more detail below. For the purpose of this disclosure, the discussion below is directed toward the treatment of a coronary artery and/or a coronary ostium and will be so described in the interest of brevity. This, however, is not intended to be limiting as the skilled person will recognize that the following discussion may also apply to another vessel and/or vessel ostium with no or minimal changes to the structure and/or scope of the disclosure. Similarly, the medical devices disclosed herein may have applications and uses in other portions of a patient's anatomy, such as but not limited to, arteries, veins, and/or other body lumens.

The figures illustrate selected components and/or arrangements of anatomy, an ostial stent delivery device, an ostial stent system, and/or methods of delivering an ostial stent. It should be noted that in any given figure, some features of the anatomy and/or the balloon valvuloplasty catheter may not be shown, or may be shown schematically, for simplicity. Additional details regarding some of the components of the anatomy, the ostial stent delivery device, and/or the ostial stent system may be illustrated in other figures in greater detail. Additionally, not all instances of some elements or features may be shown in each figure for clarity.

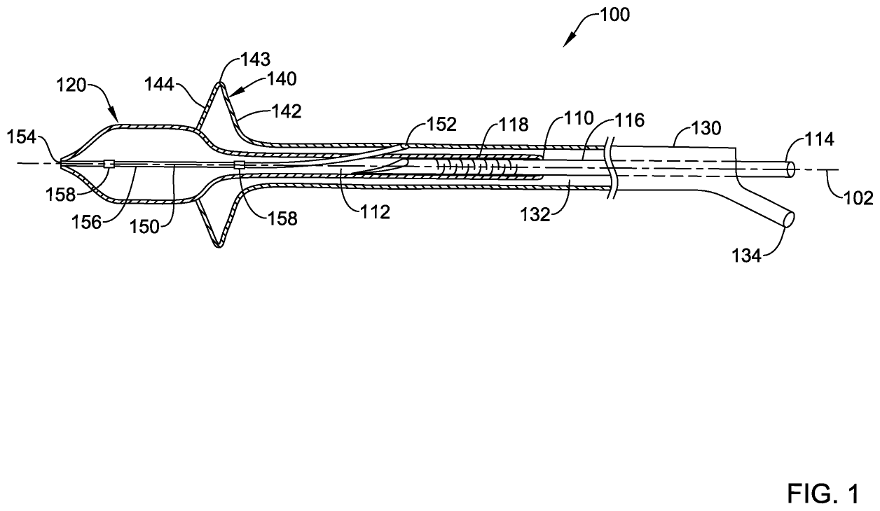

FIG. 1 is a partial cross-sectional view illustrating aspects of an ostial stent delivery device 100. The ostial stent delivery device 100 may include a first elongate shaft 110 including a first lumen 112 extending therein. The first elongate shaft 110 may extend along a central longitudinal axis 102 of the ostial stent delivery device 100 and/or the first elongate shaft 110. In some embodiments, a handle may be disposed and/or attached at a proximal end of the ostial stent delivery device 100. In some embodiments, the ostial stent delivery device 100 may include a nose cone disposed at a distal end thereof. The nose cone may be configured to slidably receive and/or slidably move over a guidewire. In at least some embodiments, the nose cone may have an atraumatic shape.

In some embodiments, the first elongate shaft 110 may include a proximal tubular member 116 and a distal tubular member 118 fixedly attached to the proximal tubular member 116. In some embodiments, the proximal tubular member 116 may be a metallic hypotube. In some embodiments, the proximal tubular member 116 may be formed from a polymeric material. In some embodiments, the distal tubular member 118 may be a polymeric tube. In some embodiments, the proximal tubular member 116 may include one or more laser cuts, strain relief cuts, perforations, and/or other features formed therein and/or at least partially through a side wall of the proximal tubular member 116 distal of a proximal end of the distal tubular member 118. In some alternative embodiments, the first elongate shaft 110 may be formed from a unitary and/or monolithic tubular member. Some suitable but non-limiting examples of materials for the first elongate shaft 110, the proximal tubular member 116, and/or the distal tubular member 118 are discussed below.

The first elongate shaft 110 may include a first inflatable balloon 120 fixedly attached thereto proximate a distal end of the first elongate shaft 110 and/or the distal tubular member 118. In some embodiments, the first inflatable balloon 120 may be integrally formed with and/or may be monolithic with the first elongate shaft 110 and/or the distal tubular member 118. In some embodiments, the first elongate shaft 110 and the first inflatable balloon 120 may be formed as separate pieces and/or elements and the first inflatable balloon 120 may then be assembled, joined, and/or attached to the first elongate shaft 110 and/or the distal tubular member 118. In some embodiments, the first inflatable balloon 120 may be melt bonded, comingled, and/or reflowed with the first elongate shaft 110 and/or the distal tubular member 118 to permanently and/or irreversibly join and/or merge the first inflatable balloon 120 with the first elongate shaft 110 and/or the distal tubular member 118.

The first inflatable balloon 120 may include a proximal waist, a distal waist, and a body portion extending between the proximal waist and the distal waist. The proximal waist of the first inflatable balloon 120 may be fixedly attached to the first elongate shaft 110 and/or the distal tubular member 118. The first inflatable balloon 120 may have a substantially cylindrical shape along a majority of its length and/or along a majority of a length of the body portion in the deployed configuration of the first inflatable balloon 120.

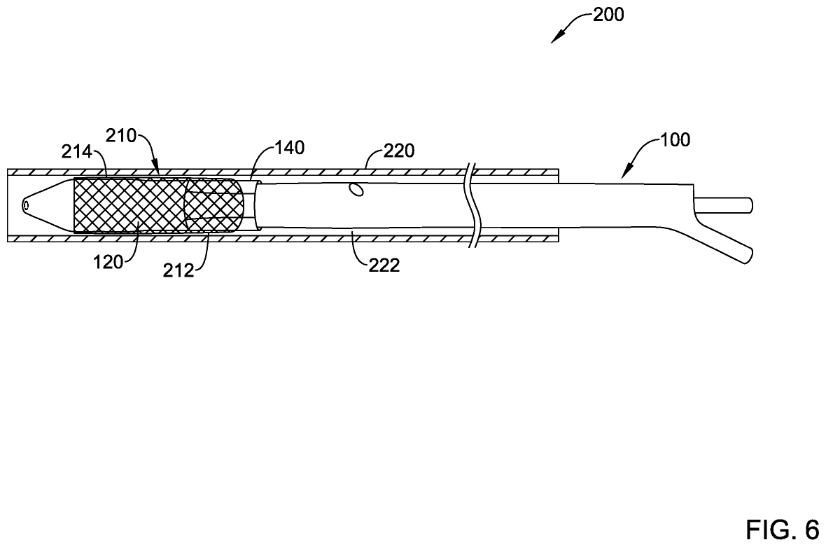

The first inflatable balloon 120 may be in fluid communication with the first lumen 112. In at least some embodiments, the first elongate shaft 110 and/or the proximal tubular member 116 may include a first proximal port 114 in fluid communication with the first lumen 112. The first proximal port 114 may be configured to receive a first inflation media for inflating and/or shifting the first inflatable balloon 120 to a deployed configuration, illustrated in FIG. 1. During delivery of an ostial stent, as described herein, the first inflatable balloon 120 may be disposed in a delivery configuration (e.g., FIG. 6) that is radially compressed and/or collapsed compared to the deployed configuration. The first inflatable balloon 120 may be selectively shifted between the delivery configuration and the deployed configuration via manipulation of the first inflation media. In some embodiments, the first inflatable balloon 120 may be formed from a compliant material. In some embodiments, the first inflatable balloon 120 may be formed from a semi-compliant material. In some embodiments, the first inflatable balloon 120 may be formed from a non-compliant material. Some suitable but non-limiting examples of materials for the first inflatable balloon 120 are discussed below.

In some embodiments, the first inflation media may include a fluid. In some embodiments, the first inflation media may include a sterile saline solution or another suitable and/or biocompatible liquid. In some embodiments, the first inflation media may include a gas. In some embodiments, the first inflation media may include air or another suitable and/or biocompatible gas. Other configurations are also contemplated. For example, in some embodiments, the first inflation media may be a gel, a semi-liquid, a slurry, and/or a mixture of substances and/or materials. In some embodiments, the first inflatable balloon 120 may be substantially impermeable to fluids and/or the first inflation media (e.g., gases, liquids, air, water, saline, blood, etc.). In some embodiments, the first inflatable balloon 120 may be semi-permeable and/or permeable to selected and/or pre-determined fluids (e.g., permeable to liquids but not gases, or vice versa, permeable to liquids but not semi-solids such as a gel, etc.). Other configurations are also contemplated.

The ostial stent delivery device 100 may include a second elongate shaft 130 including a second lumen 132 extending therein. The second elongate shaft 130 may extend along the central longitudinal axis 102 of the ostial stent delivery device 100 and/or the first elongate shaft 110. In at least some embodiments, the first elongate shaft 110 may be at least partially disposed within the second lumen 132 of the second elongate shaft 130. In some embodiments, the first elongate shaft 110 may be disposed coaxially within the second lumen 132 of the second elongate shaft 130. In some embodiments, the first elongate shaft 110 and the second elongate shaft 130 may be concentrically and/or coaxially disposed along and/or about the central longitudinal axis 102 of the ostial stent delivery device 100 and/or the first elongate shaft 110. Some suitable but non-limiting examples of materials for the second elongate shaft 130 are discussed below.

The second elongate shaft 130 may include a second inflatable balloon 140 fixedly attached thereto proximate a distal end of the second elongate shaft 130. In some embodiments, the second inflatable balloon 140 may be integrally formed with and/or may be monolithic with the second elongate shaft 130. In some embodiments, the second elongate shaft 130 and the second inflatable balloon 140 may be formed as separate pieces and/or elements and the second inflatable balloon 140 may then be assembled, joined, and/or attached to the second elongate shaft 130. In some embodiments, the second inflatable balloon 140 may be melt bonded, comingled, and/or reflowed with the second elongate shaft 130 to permanently and/or irreversibly join and/or merge the second inflatable balloon 140 with the second elongate shaft 130. The second inflatable balloon 140 may have a substantially biconical shape having a central axis oriented parallel to the central longitudinal axis 102 of the ostial stent delivery device 100 and/or the first elongate shaft 110 in the deployed configuration of the second inflatable balloon 140. For example, the second inflatable balloon 140 may include a first conical portion 142 tapering radially inward in a proximal direction to a proximal waist and a second conical portion 144 tapering radially inward in a distal direction to a distal waist. The central axis may extend from a center of the proximal waist to a center of the distal waist. In at least some embodiments, the second inflatable balloon 140, the first conical portion 142, and/or the second conical portion 144 may be centered about the central axis. A base or widened end of the first conical portion 142 may abut and/or meet a base or widened end of the second conical portion 144 to define a radially outermost extent 143 of the second inflatable balloon 140 at a medial portion of the second inflatable balloon.

The second inflatable balloon 140 may be in fluid communication with the second lumen 132. In at least some embodiments, the second elongate shaft 130 may include a second proximal port 134 in fluid communication with the second lumen 132. The second proximal port 134 may be configured to receive a second inflation media for inflating and/or shifting the second inflatable balloon 140 to a deployed configuration, illustrated in

FIG. 1. During delivery of an ostial stent, as described herein, the second inflatable balloon 140 may be disposed in a delivery configuration (e.g., FIG. 6) that is radially compressed and/or collapsed compared to the deployed configuration. The second inflatable balloon 140 may be selectively shifted between the delivery configuration and the deployed configuration via manipulation of the second inflation media. In some embodiments, the second inflatable balloon 140 may be formed from a compliant material. In some embodiments, the second inflatable balloon 140 may be formed from a semi-compliant material. In some embodiments, the second inflatable balloon 140 may be formed from a non-compliant material. In at least some embodiments, the first inflatable balloon 120 may be formed from a first material and the second inflatable balloon 140 may be formed from a second material. In some embodiments, the second material may be different from the first material. Some suitable but non-limiting examples of materials for the second inflatable balloon 140 are discussed below.

In some embodiments, the second inflation media may include a fluid. In some embodiments, the second inflation media may include a sterile saline solution or another suitable and/or biocompatible liquid. In some embodiments, the second inflation media may include a gas. In some embodiments, the second inflation media may include air or another suitable and/or biocompatible gas. Other configurations are also contemplated. For example, in some embodiments, the second inflation media may be a gel, a semi-liquid, a slurry, and/or a mixture of substances and/or materials. In some embodiments, the second inflatable balloon 140 may be substantially impermeable to fluids and/or the second inflation media (e.g., gases, liquids, air, water, saline, blood, etc.). In some embodiments, the second inflatable balloon 140 may be semi-permeable and/or permeable to selected and/or pre-determined fluids (e.g., permeable to liquids but not gases, or vice versa, permeable to liquids but not semi-solids such as a gel, etc.). Other configurations are also contemplated.

As seen in FIG. 1, the second inflatable balloon 140 may be disposed at least partially proximal of the first inflatable balloon 120. In at least some embodiments, the second inflatable balloon 140 and/or the distal waist of the second inflatable balloon 140 may be fixedly attached and/or directly attached to the first inflatable balloon 120. In some embodiments, the second inflatable balloon 140 and/or the distal waist of the second inflatable balloon 140 may be fixedly attached and/or directly attached to the body portion of the first inflatable balloon 120. In some embodiments, the second inflatable balloon 140 and/or the distal waist of the second inflatable balloon 140 may be adhesively bonded to the first inflatable balloon 120 and/or to the body portion of the first inflatable balloon 120. In some embodiments, the second inflatable balloon 140 and/or the distal waist of the second inflatable balloon 140 may be welded (e.g., laser welded, sonic welded, etc.) to the first inflatable balloon 120 and/or to the body portion of the first inflatable balloon 120. In some embodiments, the second inflatable balloon 140 may be oriented coaxially with the first inflatable balloon 120.

The second inflatable balloon 140 may have a radially outermost extent 143 in the deployed configuration of the second inflatable balloon 140 that is greater than a radially outermost extent of the first inflatable balloon 120 and/or of the body portion of the first inflatable balloon 120 in the deployed configuration of the first inflatable balloon 120. For example, the second inflatable balloon 140 may include a substantially biconical body portion having the radially outermost extent 143 in the deployed configuration of the second inflatable balloon 140 that is greater than the radially outermost extent of a substantially cylindrical body portion of the first inflatable balloon 120 in the deployed configuration of the first inflatable balloon 120.

The ostial stent delivery device 100 may include a guidewire lumen 150 extending from a proximal guidewire port 152 disposed in and/or through a wall of the second elongate shaft 130 to a distal guidewire port 154 at a distal end of the first inflatable balloon 120 and/or the ostial stent delivery device 100. It at least some embodiments, the guidewire lumen 150 may be defined and/or formed by a guidewire tube 156 extending from the proximal guidewire port 152 to the distal guidewire port 154. The distal waist of the first inflatable balloon 120 may be fixedly attached to a distal end portion of the guidewire tube 156. In some embodiments, a distal portion of the guidewire tube 156 may be coaxial with the central longitudinal axis 102 of the ostial stent delivery device 100 and/or the first elongate shaft 110. In at least some embodiments, the ostial stent delivery device 100 may be configured for use in a monorail, rapid exchange, or single operator exchange configuration. In some alternative configurations, the ostial stent delivery device 100 may be configured for use in an over-the-wire configuration. In an over-the-wire design, a tri-lumen tubing may be used, wherein one of the lumens defines a guidewire lumen extending proximally to a 3-way manifold at the proximal end of the ostial stent delivery device 100. The other two lumens in the tri-lumen tubing may be used to control inflation of the first inflatable balloon 120 and the second inflatable balloon 140. Other configurations are also contemplated.

In some embodiments, the guidewire tube 156 may include at least one radiopaque marker 158 disposed along and/or secured to the guidewire tube 156. In some embodiments, the guidewire tube 156 may include at least one radiopaque marker 158 disposed radially inward of and/or within the first inflatable balloon 120 and/or the second inflatable balloon 140. In some embodiments, the at least one radiopaque marker 158 may include one radiopaque marker, two radiopaque markers, three radiopaque markers, four radiopaque markers, or another number of radiopaque markers. In some embodiments, the at least one radiopaque marker 158 may include a first radiopaque marker and a second radiopaque marker spaced longitudinally apart from the first radiopaque marker. In some embodiments, the at least one radiopaque marker 158 may be disposed at and/or adjacent to a proximal end and/or a distal end of the body portion of the first inflatable balloon 120. In some embodiments, the at least one radiopaque marker 158 may be fixedly attached to the guidewire tube 156. In some embodiments, the at least one radiopaque marker 158 may be embedded within the guidewire tube 156. Other configurations are also contemplated. Some suitable but non-limiting examples of materials for the guidewire tube 156, the at least one radiopaque marker 158, etc. are discussed below.

In some embodiments, the second elongate shaft 130 may include and/or may form a seal around the first elongate shaft 110 proximate the first proximal port 114. For example, the first elongate shaft 110 may extend proximal of the second elongate shaft 130 to the first proximal port 114. The seal may prevent the second inflation media from escaping the second lumen 132 and/or the seal may prevent contamination of the second lumen 132. Other configurations are also contemplated.

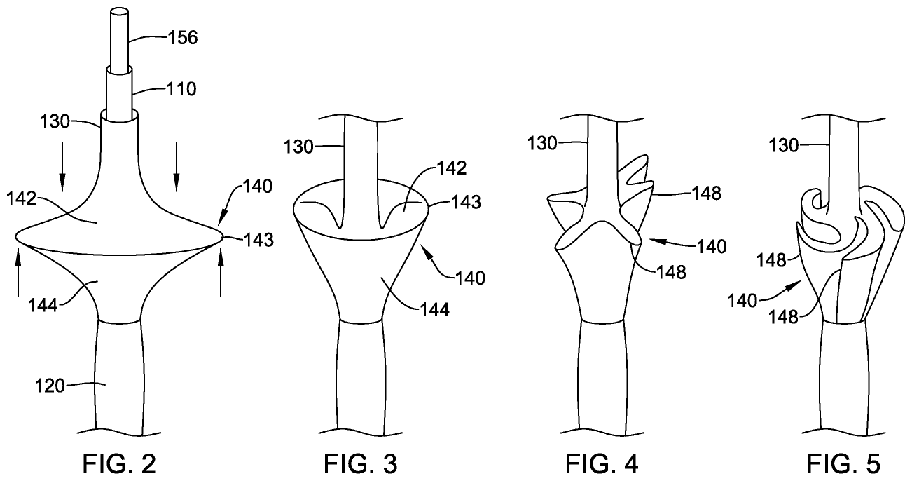

FIGS. 2-5 are partial cutaway views illustrating an example method or process for collapsing and/or folding the second inflatable balloon 140 to the delivery configuration, which may be suitable for and/or may be configured to receive an ostial stent secured thereto in a collapsed configuration. Some elements of the figures are labeled for reference but are not expressly discussed herein.

FIG. 2 illustrates the first inflatable balloon 120 in the delivery configuration and/or substantially collapsed. After initially forming the second inflatable balloon 140, and/or as the second inflation media is removed from the second inflatable balloon 140, the second inflatable balloon 140 may be shifted from the deployed configuration to the delivery configuration. When disposing the second inflatable balloon 140 in the delivery configuration, the radially outermost extent 143 of the second inflatable balloon 140 may be urged proximally and a proximal portion (e.g., the first conical portion 142) of the second inflatable balloon 140 disposed proximal of the radially outermost extent 143 of the second inflatable balloon 140 may be urged distally. For example, a proximal force may be applied to an outer portion of the second conical portion 144 and/or to the radially outermost extent 143 of the second inflatable balloon 140 as a distal force is applied to an inner portion of the first conical portion 142, as illustrated with arrows in FIG. 2. This will cause the radially outermost extent 143 of the second inflatable balloon 140 to shift proximally over and/or radially outward of the first conical portion 142 of the second inflatable balloon 140, as seen in FIG. 3. In some embodiments, as the radially outermost extent 143 of the second inflatable balloon 140 is shifted proximally and/or to the delivery configuration, at least a portion of the second inflatable balloon 140 may be disposed proximal of the of the proximal waist of the second inflatable balloon 140. Next, the second inflatable balloon 140 may be folded into pleats 148, as seen in FIG. 4. Finally, the pleats 148 may be wrapped around the second elongate shaft 130 and/or the first elongate shaft 110 disposed radially inward of the second inflatable balloon 140, as shown in FIG. 5. An ostial stent may subsequently be secured to the first inflatable balloon 120 and the second inflatable balloon 140 in a collapsed configuration for delivery to a treatment site. For example, the ostial stent may be crimped onto the first inflatable balloon 120 and the second inflatable balloon 140. In another example, the ostial stent may be adhered to the first inflatable balloon 120 and the second inflatable balloon 140. Other configurations are also contemplated.

FIG. 6 illustrates selected aspects of an ostial stent system 200. The ostial stent system 200 may include the ostial stent delivery device 100 as described herein. The ostial stent system 200 may include an ostial stent 210 having a proximal portion 212 and a distal portion 214. The ostial stent system 200 may include a guide catheter 220 having a lumen 222 extending therethrough. The ostial stent system 200 is shown in FIG. 6 with the guide catheter 220 in cross-section to improve clarity. The ostial stent delivery device 100 may be movably disposed within the lumen 222 of the guide catheter 220. In some embodiments, the ostial stent delivery device 100 may be slidably and/or rotatably disposed within the lumen 222 of the guide catheter 220. In some embodiments, the ostial stent delivery device 100 (including the ostial stent 210 disposed thereon) may be configured to be slidably and/or rotatably disposed within a guide catheter 220 having an outer diameter of about 4 French (Fr) to about 8 Fr. Other configurations are also contemplated. For example, in some embodiments, the ostial stent delivery device 100 (including the ostial stent 210 disposed thereon) may be configured to be slidably and/or rotatably disposed within a guide catheter 220 having an outer diameter of about 4 Fr to about 8 Fr.

The distal portion 214 of the ostial stent 210 may be secured to the first inflatable balloon 120 in a collapsed configuration when the first inflatable balloon 120 is disposed within the lumen 222 of the guide catheter 220 and/or when the first inflatable balloon 120 is in the delivery configuration. The proximal portion 212 of the ostial stent 210 may be secured to the second inflatable balloon 140 in a collapsed configuration when the second inflatable balloon 140 is disposed within the lumen 222 of the guide catheter 220 and/or when the second inflatable balloon 140 is in the delivery configuration.

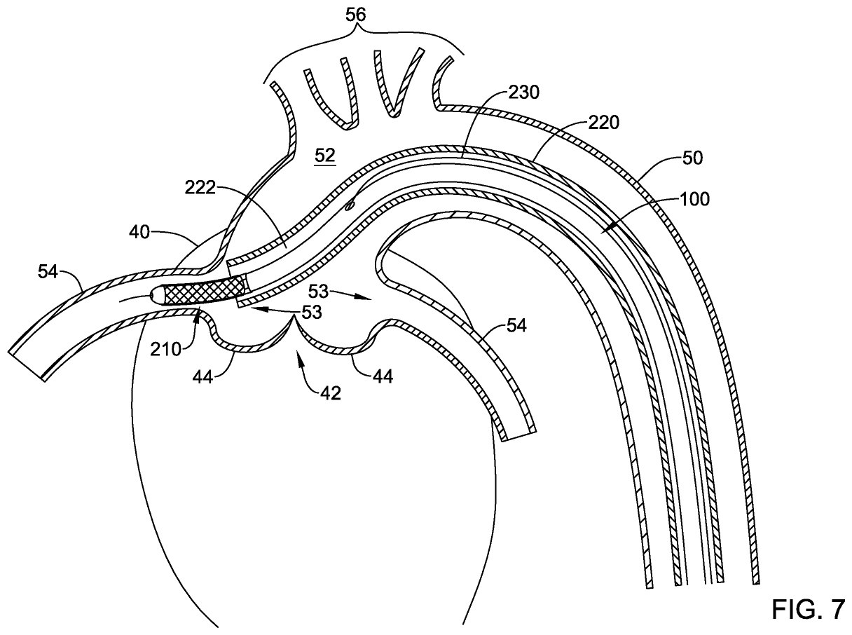

FIGS. 7-10 illustrate aspects of a method of delivering the ostial stent 210. The figures schematically illustrate in a partial cut-away view aspects of a patient's heart 40, including an aortic valve 42 having valve leaflets 44, and certain connected vasculature, such as the aorta 50 connected to the aortic valve 42 of the heart 40 by the aortic arch 52, the coronary artery 54, the ostium 53 of the coronary artery 54, and other large arteries 56 (e.g., subclavian arteries, carotid arteries, brachiocephalic artery) that extend from the aortic arch 52 to important internal organs. For the purpose of this disclosure, the discussion below is directed toward use in the coronary artery 54 and/or the ostium 53 of the coronary artery 54 and will be so described in the interest of brevity. This, however, is not intended to be limiting as the skilled person will recognize that the following discussion may also apply to other ostia, vessels, and/or treatment locations within a patient with no or minimal changes to the structure and/or scope of the disclosure.

The method may include advancing the guide catheter 220 intravascularly to the ostium 53 of a vessel (e.g., the coronary artery 54, etc.). The ostial stent delivery device 100 may be advanced over a guidewire 230 positioned in and/or extending through the guidewire lumen 150. The guidewire 230 may have been previously positioned within the treatment site and/or the ostium 53 of the vessel (e.g., the coronary artery 54, etc.). In at least some embodiments, a distal end of the guidewire 230 may be positioned within the vessel (e.g., the coronary artery 54, etc.).

The ostial stent delivery device 100 and the ostial stent 210 may be moved out of the lumen 222 of the guide catheter 220 proximate and/or into the ostium 53 of the vessel (e.g., the coronary artery 54, etc.). In some embodiments, the ostial stent delivery device 100 and the ostial stent 210, in the collapsed configuration, may be positioned within the ostium 53 of the vessel (e.g., the coronary artery 54, etc.) and/or within the vessel itself. For example, in some procedures, the ostial stent 210 may only be used if necessary or if a problem is encountered during another procedure (e.g., a protected TAVR procedure, etc.). As such, the ostial stent 210, in the collapsed configuration, may be positioned on the ostial stent delivery device 100 within the vessel for later use. In some embodiments, the ostial stent delivery device 100 and the ostial stent 210 may be used immediately to treat the ostium 53 of the vessel (e.g., the coronary artery 54, etc.) upon delivery thereto.

The method may include positioning the proximal portion 212 of the ostial stent 210, in the collapsed configuration, such that the proximal portion 212 of the ostial stent 210 is spaced apart proximal of the ostium 53 of the vessel (e.g., the coronary artery 54, etc.), as seen in FIG. 7. For example, the proximal portion 212 of the ostial stent 210 may be positioned within the aortic arch 52 and spaced apart from the ostium 53 of the vessel (e.g., the coronary artery 54, etc.). In some embodiments, this may be done with or without the guide catheter 220 in place proximate the ostium 53 of the vessel (e.g., the coronary artery 54, etc.).

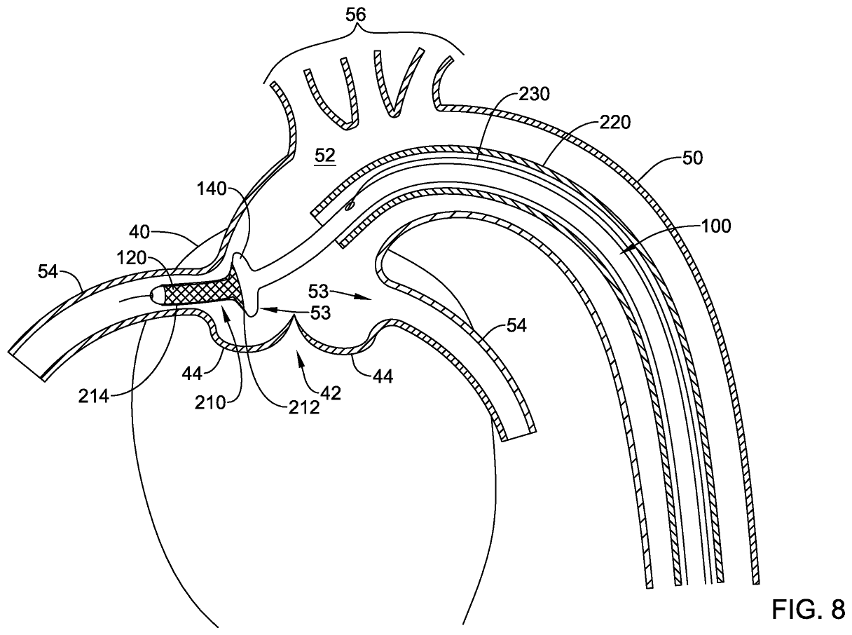

The method may include expanding the proximal portion 212 of the ostial stent 210 to the expanded configuration while maintaining the distal portion 214 of the ostial stent 210 in the collapsed configuration, wherein the proximal portion 212 of the ostial stent 210 remains spaced apart from the ostium 53 of the vessel (e.g., the coronary artery 54, etc.), as seen in FIG. 8. The second inflatable balloon 140 may be configured to expand the proximal portion 212 of the ostial stent 210 when the second inflatable balloon 140 is shifted from the delivery configuration to the deployed configuration. As such, expanding the proximal portion 212 of the ostial stent 210 may be done by inflating and/or shifting the second inflatable balloon 140 of the ostial stent delivery device 100 to the deployed configuration. For example, the second inflation media may be passed through the second lumen 132 of the second elongate shaft 130 from the second proximal port 134 of the second elongate shaft 130 to the second inflatable balloon 140. In the expanded configuration, the proximal portion 212 of the ostial stent 210 may have a substantially conical shape expanding radially outward in a proximal direction from the distal portion 214 of the ostial stent 210.

In at least some embodiments, expanding the proximal portion 212 of the ostial stent 210 may include inflating the second inflatable balloon 140 of the ostial stent delivery device 100 without inflating the first inflatable balloon 120 of the ostial stent delivery device 100. In some embodiments, the first inflatable balloon 120 is inflatable independently of the second inflatable balloon 140 and/or the second inflatable balloon 140 is inflatable independently of the first inflatable balloon 120. In some embodiments, when the second inflatable balloon 140 is inflated and/or shifted to the deployed configuration, at least a portion of the first inflatable balloon 120 extends proximal of and/or is positioned outside of the ostium 53 of the vessel (e.g., the coronary artery 54, etc.). Accordingly, when the first inflatable balloon 120 and the second inflatable balloon 140 are disposed outside of the lumen 222 of the guide catheter 220 in a first position, the distal portion 214 of the ostial stent 210 is disposed in the collapsed configuration and the proximal portion 212 of the ostial stent 210 is disposed in the expanded configuration. In some embodiments, prior to expanding the proximal portion 212 of the ostial stent 210 and/or prior to inflating and/or shifting the second inflatable balloon 140 to the deployed configuration, the distal portion 214 of the ostial stent 210 may be secured to the first inflatable balloon 120 in the collapsed configuration and the proximal portion 212 of the ostial stent 210 may be secured to the second inflatable balloon 140 in the collapsed configuration.

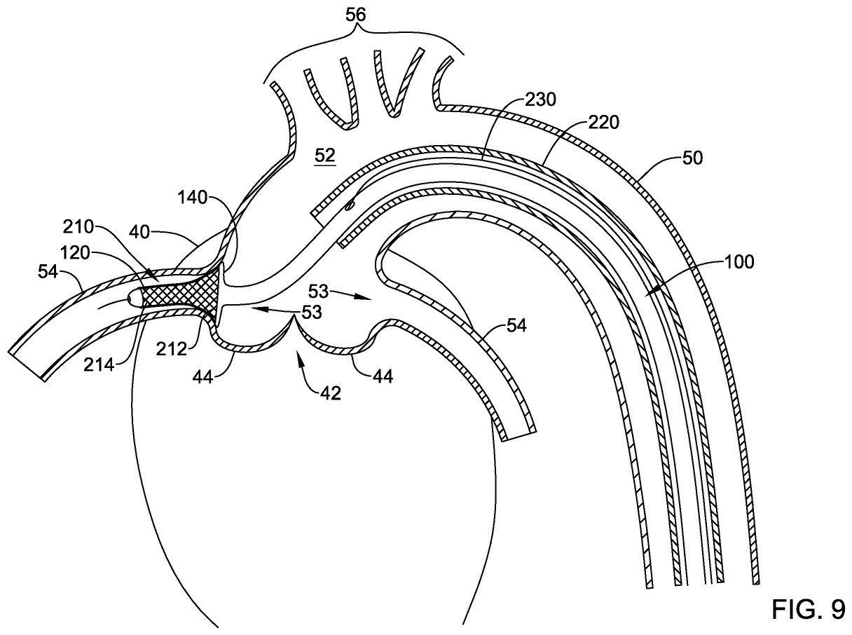

The method may include advancing the distal portion 214 of the ostial stent 210 in the collapsed configuration and/or advancing the first inflatable balloon 120 in the delivery configuration into the vessel (e.g., the coronary artery 54, etc.) until at least part of the proximal portion 212 of the ostial stent 210, in the expanded configuration, engages the ostium 53 of the vessel (e.g., the coronary artery 54, etc.), as seen in FIG. 9. Advancing the distal portion 214 of the ostial stent 210 into the vessel in the collapsed configuration with the proximal portion 212 of the ostial stent 210 in the expanded configuration may permit the ostial stent 210 to accurately locate and/or engage the ostium 53 of the vessel (e.g., the coronary artery 54, etc.).

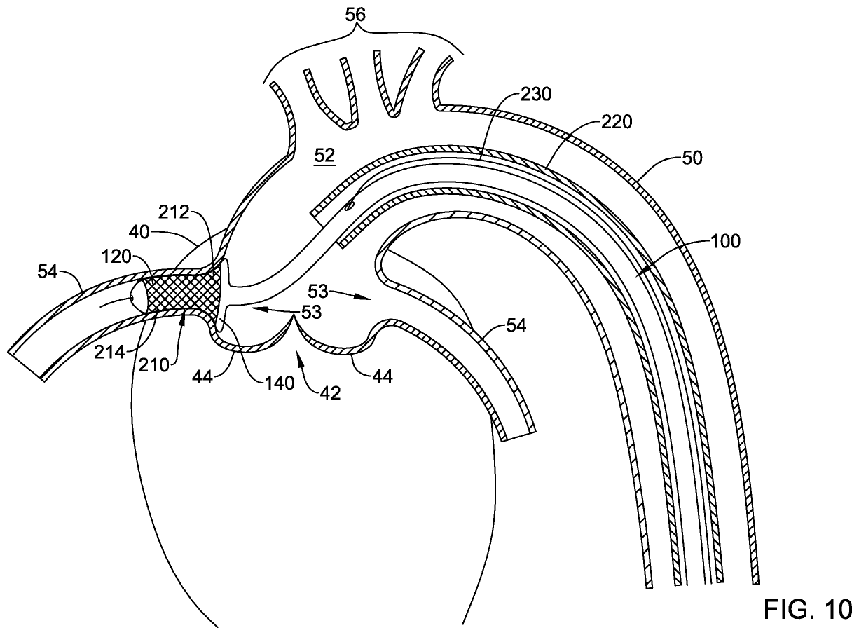

The method may include expanding the distal portion 214 of the ostial stent 210 to the expanded configuration within the vessel (e.g., the coronary artery 54, etc.) to engage the distal portion 214 of the ostial stent 210 against a wall of the vessel, as seen in FIG. 10. The first inflatable balloon 120 may be configured to expand the distal portion 214 of the ostial stent 210 when the first inflatable balloon 120 is inflated and/or shifted from the delivery configuration to the deployed configuration. As such, expanding the distal portion 214 of the ostial stent 210 may be done by inflating and/or shifting the first inflatable balloon 120 of the ostial stent delivery device 100 to the deployed configuration. For example, the first inflation media may be passed through the first lumen 112 of the first elongate shaft 110 from the first proximal port 114 of the first elongate shaft 110 to the first inflatable balloon 120. In the expanded configuration, the distal portion 214 of the ostial stent 210 may have a substantially cylindrical shape extending in a distal direction from the proximal portion 212 of the ostial stent 210.

In at least some embodiments, expanding the distal portion 214 of the ostial stent 210 may include inflating the first inflatable balloon 120 of the ostial stent delivery device 100 after inflating the first inflatable balloon 120 of the ostial stent delivery device 100. In some embodiments, when the first inflatable balloon 120 is inflated and/or shifted to the deployed configuration, the first inflatable balloon 120 extends distal of the ostium 53 of the vessel and/or is positioned inside of the vessel (e.g., the coronary artery 54, etc.). Accordingly, when the first inflatable balloon 120 and the second inflatable balloon 140 are disposed outside of the lumen 222 of the guide catheter 220 in a second position, the distal portion 214 of the ostial stent 210 is shiftable to the expanded configuration upon inflation of the first inflatable balloon 120.

After expanding the distal portion 214 of the ostial stent 210 within the vessel (e.g., the coronary artery 54, etc.), the first inflatable balloon 120 may be deflated and/or shifted from the deployed configuration to the delivery configuration by removing the first inflation media from the first inflatable balloon 120 via the first lumen 112 of the first elongate shaft 110. After expanding the distal portion 214 of the ostial stent 210 within the vessel (e.g., the coronary artery 54, etc.), the second inflatable balloon 140 may be deflated and/or shifted from the deployed configuration toward the delivery configuration by removing the second inflation media from the second inflatable balloon 140 via the second lumen 132 of the second elongate shaft 130. The ostial stent delivery device 100, with the first inflatable balloon 120 and the second inflatable balloon 140 in a deflated state, may be withdrawn into the lumen 222 of the guide catheter 220. The ostial stent delivery device 100 and/or the guide catheter 220 may then be removed from the patient's vasculature.

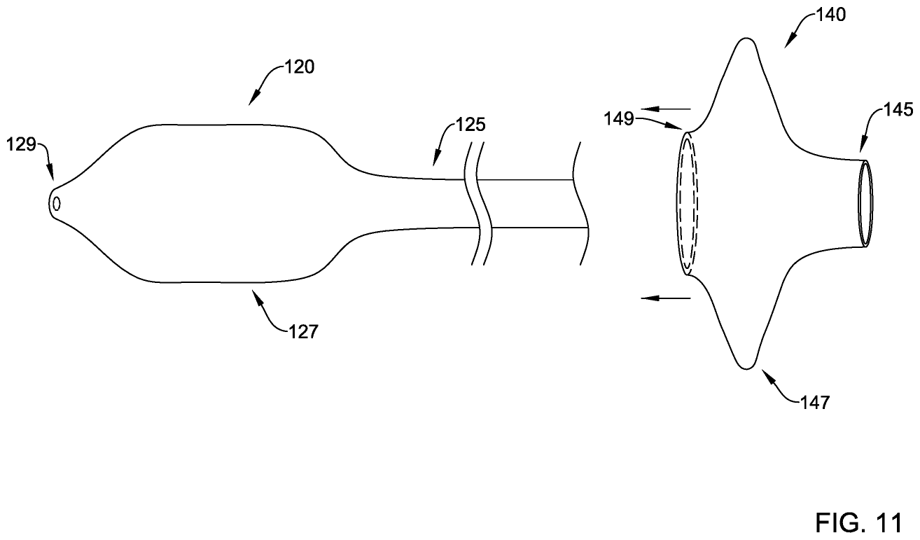

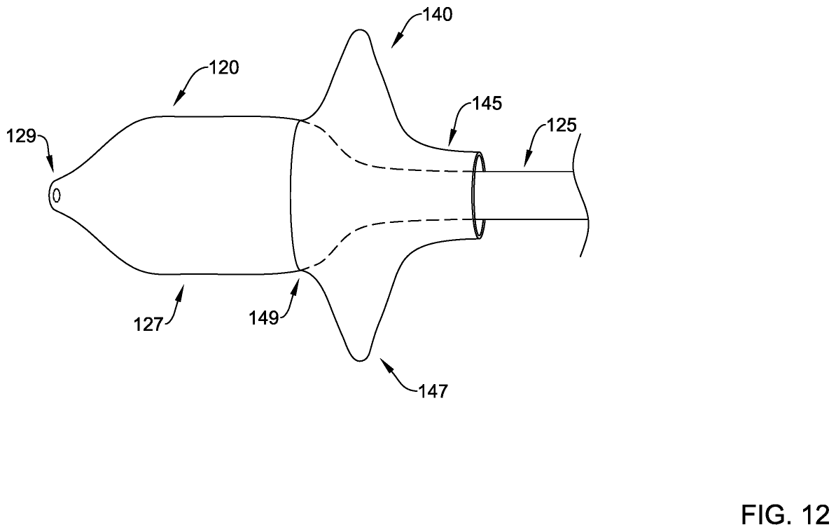

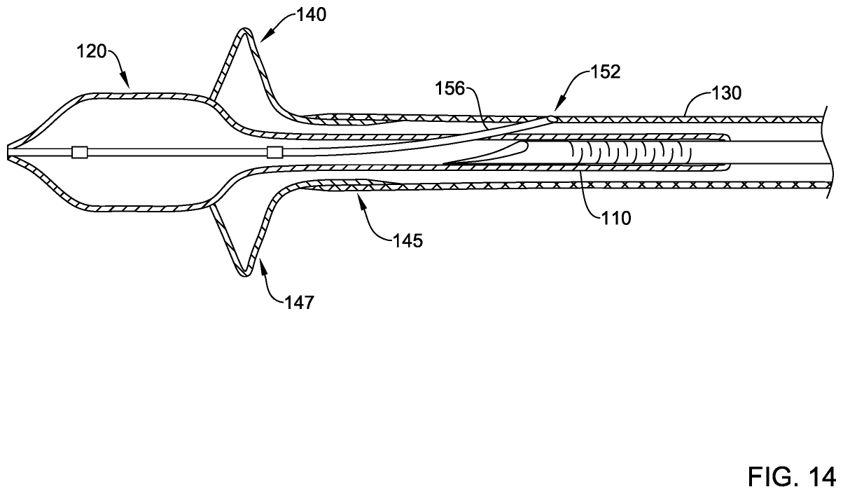

FIGS. 11-14 illustrate selected aspects of a method of manufacturing an ostial stent delivery device. The method may include forming the first inflatable balloon 120, wherein in the deployed configuration (and/or an inflated configuration) the first inflatable balloon 120 includes a first proximal waist 125, a substantially cylindrical body portion 127, and a first distal waist 129. The method may include forming the second inflatable balloon 140, wherein in the deployed configuration (and/or an inflated configuration) the second inflatable balloon 140 includes a second proximal waist 145, a substantially biconical body portion 147, and a second distal waist 149.

In some embodiments, forming the first inflatable balloon 120 and/or the second inflatable balloon 140 may include one or more methods or processes. In some embodiments, forming the first inflatable balloon 120 and/or the second inflatable balloon 140 may include blow molding, injection molding, casting, etc. In some embodiments, forming the first inflatable balloon 120 may include forming separate pieces of the first inflatable balloon 120 and then joining those pieces together to form the first inflatable balloon 120. In some embodiments, forming the second inflatable balloon 140 may include forming separate pieces of the second inflatable balloon 140 and then joining those pieces together to form the second inflatable balloon 140. Other configurations and/or processes are also contemplated.

In some embodiments, the second distal waist 149 may have a diameter greater than a diameter of the second proximal waist 145 and/or a diameter of the first proximal waist 125. In some embodiments, the diameter of the second proximal waist 145 may be greater than the diameter of the first proximal waist 125. The substantially biconical body portion 147 of the second inflatable balloon 140 may have a radially outermost extent in the deployed configuration (and/or the inflated configuration) of the second inflatable balloon 140 that is greater than a radially outermost extent of the substantially cylindrical body portion 127 of the first inflatable balloon 120 in the deployed configuration (and/or the inflated configuration) of the first inflatable balloon 120.

The method may include positioning and/or advancing the second distal waist 149 of the second inflatable balloon 140 over and/or relative to the first proximal waist 125 of the first inflatable balloon 120, as seen in FIG. 11. The method may include fixedly attaching the second distal waist 149 to the first inflatable balloon 120. In at least some embodiments, the method may include fixedly attaching the second distal waist 149 to the substantially cylindrical body portion 127 of the first inflatable balloon 120. The first proximal waist 125 of the first inflatable balloon 120 may extend through the second inflatable balloon 140 and the first proximal waist 125 of the first inflatable balloon 120 may extend proximal of the second proximal waist 145 of the second inflatable balloon 140.

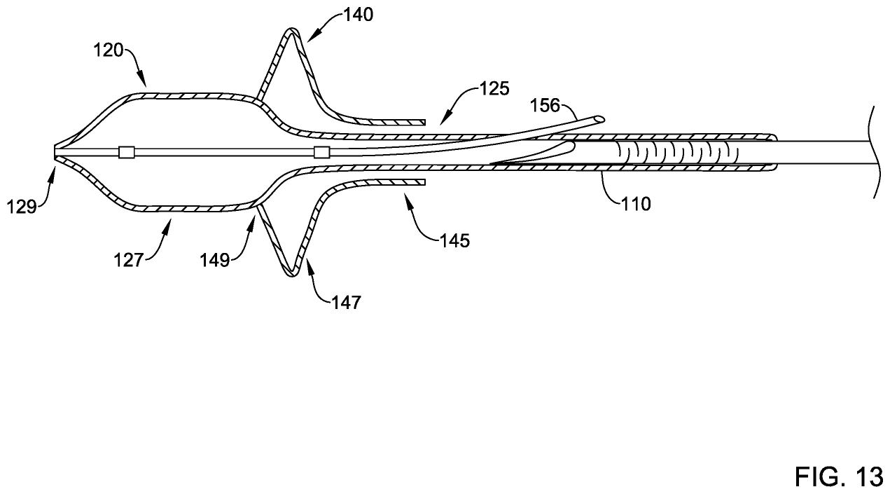

The method may include fixedly attaching the first proximal waist 125 of the first inflatable balloon 120 to a distal end of a first elongate shaft 110, wherein the first elongate shaft 110 includes a first lumen extending therein. In some embodiments, the first proximal waist 125 may be bonded, welded, and/or melted together with the distal end of the first elongate shaft 110. In some embodiments, a first mandrel may be disposed within the first lumen of the first elongate shaft 110 prior to fixedly attaching the first proximal waist 125 of the first inflatable balloon 120 to the distal end of the first elongate shaft 110. In some embodiments, the first inflatable balloon 120 may be integrally formed with and/or from the first elongate shaft 110. Other configurations are also contemplated. The method may include disposing a guidewire tube 156 through a wall of the first elongate shaft 110. The guidewire tube 156 may be fixedly attached to the wall of the first elongate shaft 110. A proximal portion of the guidewire tube 156 may extend outside of the first elongate shaft 110 in a proximal direction. A distal portion of the guidewire tube 156 may extend distal of the distal end of the first elongate shaft 110, such that the guidewire tube 156 extends distally through the first inflatable balloon 120. The method may include fixedly attaching the first distal waist 129 to the distal portion of the guidewire tube 156, as shown in FIG. 13.

The method may include fixedly attaching the second proximal waist 145 to a distal end of a second elongate shaft 130, wherein the second elongate shaft 130 includes a second lumen extending therein. In some embodiments, the second proximal waist 145 may be bonded, welded, and/or melted together with the distal end of the second elongate shaft 130. In some embodiments, a second mandrel may be disposed within the second lumen of the second elongate shaft 130 prior to fixedly attaching the second proximal waist 145 to the distal end of the second elongate shaft 130. The guidewire tube 156 may be disposed through a wall of the second elongate shaft 130. The proximal portion and/or a proximal end of the guidewire tube 156 may be fixedly attached to the wall of the second elongate shaft 130 for form a proximal guidewire port 152.

The materials that can be used for the various components of the ostial stent system (and/or other elements disclosed herein) and the various components thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the ostial stent, the elongate shaft(s), the first and/or second inflatable balloon(s), the first and/or second proximal port(s), the first and second radiopaque markers, the guidewire, the guide catheter, etc. and/or elements or components thereof.

In some embodiments, the system and/or other elements disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

In at least some embodiments, portions or all of the system and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the system and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or other elements disclosed herein to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MM image. The system or portions thereof, may also be made from a material that the MM machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An ostial stent delivery device, comprising:
a first elongate shaft including a first lumen extending therein, the first elongate shaft extending along a central longitudinal axis;
wherein the first elongate shaft includes a first inflatable balloon fixedly attached thereto proximate a distal end of the first elongate shaft, the first inflatable balloon being in fluid communication with the first lumen; and
a second elongate shaft including a second lumen extending therein, the first elongate shaft being at least partially disposed within the second lumen;
wherein the second elongate shaft includes a second inflatable balloon fixedly attached thereto proximate a distal end of the second elongate shaft, the second inflatable balloon being in fluid communication with the second lumen;
wherein the second inflatable balloon is disposed at least partially proximal of the first inflatable balloon;
wherein the first inflatable balloon is configured to expand a distal portion of an ostial stent when the first inflatable balloon is shifted from a delivery configuration to a deployed configuration;
wherein the second inflatable balloon is configured to expand a proximal portion of the ostial stent when the second inflatable balloon is shifted from a delivery configuration to a deployed configuration;
wherein the first inflatable balloon has a substantially cylindrical shape along a majority of its length in the deployed configuration of the first inflatable balloon;
wherein the second inflatable balloon has a substantially biconical shape having a central axis oriented parallel to the central longitudinal axis in the deployed configuration of the second inflatable balloon.

2. The ostial stent delivery device of claim 1, wherein when disposing the second inflatable balloon in the delivery configuration, a radially outermost extent of the second inflatable balloon is urged proximally and a proximal portion of the second inflatable balloon disposed proximal of the radially outermost extent of the second inflatable balloon is urged distally.

3. The ostial stent delivery device of claim 1, wherein the second inflatable balloon is fixedly attached to the first inflatable balloon.

4. The ostial stent delivery device of claim 3, wherein the second inflatable balloon is adhesively bonded to the first inflatable balloon.

5. The ostial stent delivery device of claim 3, wherein the second inflatable balloon is welded to the first inflatable balloon.

6. The ostial stent delivery device of claim 1, wherein the first elongate shaft includes a first proximal port in fluid communication with the first lumen and configured to receive a first inflation media for inflating the first inflatable balloon to the deployed configuration.

7. The ostial stent delivery device of claim 1, wherein the second elongate shaft includes a second proximal port in fluid communication with the second lumen and configured to receive a second inflation media for inflating the second balloon to the deployed configuration.

8. The ostial stent delivery device of claim 1, wherein the second inflatable balloon is oriented coaxially with the first inflatable balloon.

9. The ostial stent delivery device of claim 1, wherein the first elongate shaft is disposed coaxially within the second lumen of the second elongate shaft.

10. An ostial stent system, comprising:
a guide catheter having a lumen extending therethrough;
an ostial stent having a proximal portion and a distal portion; and
an ostial stent delivery device slidably disposed within the lumen of the guide catheter, wherein the ostial stent delivery device comprises:
a first elongate shaft extending along a central longitudinal axis, the first elongate shaft having a first inflatable balloon fixedly attached thereto proximate a distal end of the first elongate shaft; and
a second elongate shaft extending along the central longitudinal axis, the second elongate shaft having a second inflatable balloon fixedly attached thereto proximate a distal end of the second elongate shaft;
wherein the second inflatable balloon is disposed at least partially proximal of the first inflatable balloon;
wherein the distal portion of the ostial stent is secured to the first inflatable balloon in a collapsed configuration when the first inflatable balloon is disposed within the lumen of the guide catheter;
wherein the proximal portion of the ostial stent is secured to the second inflatable balloon in a collapsed configuration with the second inflatable balloon is disposed within the lumen of the guide catheter;
wherein when the first inflatable balloon and the second inflatable balloon are disposed outside of the lumen of the guide catheter in a first position, the distal portion of the ostial stent is in the collapsed configuration and the proximal portion of the ostial stent is in an expanded configuration.

11. The ostial stent system of claim 10, wherein when the first inflatable balloon and the second inflatable balloon are disposed outside of the lumen of the guide catheter in a second position, the distal portion of the ostial stent is shiftable to an expanded configuration upon inflation of the first inflatable balloon.

12. The ostial stent system of claim 10, wherein the first inflatable balloon is inflatable independently of the second inflatable balloon.

13. The ostial stent system of claim 10, wherein the first inflatable balloon is formed from a first material and the second inflatable balloon is formed from a second material.

14. The ostial stent system of claim 10, wherein in the expanded configuration, the proximal portion of the ostial stent has a substantially conical shape expanding radially outward in a proximal direction.

15. A method of delivering an ostial stent, comprising:
positioning a proximal portion of the ostial stent such that the proximal portion is the ostial stent is spaced apart proximal of an ostium of a vessel;
expanding the proximal portion of the ostial stent to an expanded configuration while maintaining a distal portion of the ostial stent in a collapsed configuration, wherein the proximal portion of the ostial stent remains spaced apart from the ostium of the vessel;
advancing the distal portion of the ostial stent into the vessel until the proximal portion of the ostial stent engages the ostium of the vessel; and
expanding the distal portion of the ostial stent to an expanded configuration within the vessel to engage the distal portion of the ostial stent against a wall of the vessel.

16. The method of claim 15, wherein prior to expanding the proximal portion of the ostial stent, the distal portion of the ostial stent is secured to a first inflatable balloon in the collapsed configuration and the proximal portion of the ostial stent is secured to a second inflatable balloon in a collapsed configuration.

17. The method of claim 16, wherein expanding the proximal portion of the ostial stent includes inflating the second inflatable balloon without inflating the first inflatable balloon.

18. The method of claim 17, wherein when the second inflatable balloon is inflated, at least a portion of the first inflatable balloon extends proximal of the ostium of the vessel.

19. The method of claim 16, wherein expanding the distal portion of the ostial stent includes inflating the first inflatable balloon.

20. The method of claim 16, wherein the second inflatable balloon includes a substantially biconical body portion having a radially outermost extent in a deployed configuration of the second inflatable balloon that is greater than a radially outermost extent of a substantially cylindrical body portion of the first inflatable balloon in a deployed configuration of the first inflatable balloon.
